(19) United States
(12) Patent Application Publication
(10) Pub. No.: US 2012/0179039 A1
(43) Pub. Date: Jul. 12, 2012

(54) METHODS AND APPARATUS FOR PRODUCING VIDEO RECORDS OF USE OF MEDICAL ULTRASOUND IMAGING SYSTEMS

(76) Inventors: Laurent Pelissier, North Vancouver (CA); Tomas Bobovsky, Coquitlam (CA); Trevor Hansen, Vancouver (CA)

(21) Appl. No.: 13/345,623

(22) Filed: Jan. 6, 2012

Related U.S. Application Data
(60) Provisional application No. 61/430,806, filed on Jan. 7, 2011.

(51) Int. Cl. A61B 8/14; H04N 7/26

(52) U.S. Cl. 600/443; 375/240.01; 375/E07.026

(57) ABSTRACT

Methods and apparatus for producing a video record of a use of a medical ultrasound system are provided. An ultrasound image video signal and a synthetic display element signal produced by an ultrasound system are encoded as at least one data stream. The at least one data stream is stored in a multimedia container file. The ultrasound video signal and synthetic display element signal may be encoded as different data streams. In some embodiments, the ultrasound video signal and synthetic display element signal are combined in a single video signal, which is encoded as a video data stream. In some embodiments, a subject image video signal is produced by a camera, and this signals is encoded and stored with an associated ultrasound image video signal in a video container.

### CROSS-REFERENCE TO RELATED APPLICATIONS

This application claims the benefit under 35 U.S.C. §119 of U.S. Patent Application No. 61/430,806 filed on 7 Jan. 2011 and entitled METHODS AND APPARATUS FOR PRODUCING VIDEO RECORDS OF USE OF MEDICAL ULTRASOUND IMAGING SYSTEMS which is incorporated hereby by reference.

### TECHNICAL FIELD

The invention relates to methods and apparatus for producing records of use of medical ultrasound imaging systems. Some embodiments produce records containing ultrasound image video signals and synthetic display element signals conveying information that may be displayed to an ultrasound technician during use of medical ultrasound imaging systems.

### BACKGROUND

Ultrasound examinations may performed on animals, such as humans, to acquire images useful in the treatment or diagnosis of a wide range of medical conditions. In ultrasound imaging, ultrasound pulses are transmitted into a body, and reflected off of structures in the body (e.g., interfaces where there is a density change in the body). Reflected ultrasound pulses (echos) are detected at a transducer. Timing and strength information for reflected pulses is used to construct images. Two-dimensional ultrasound images show a cross-sectional “slice” of anatomy traversed by the ultrasound pulses.

Some ultrasound apparatus comprise freehand ultrasound probes, which ultrasound technicians may move freely over the skin of a subject to acquire images of different slices of the subject's anatomy. An ultrasound technician may maneuver such probes to acquire images that show particular views of particular structures (e.g., views of structures prescribed by a physician).

Ultrasound images may be analyzed by medical personnel, such as radiologists, physicians and the like. Typically, ultrasound images are provided to medical personnel with indications of the anatomical structure(s) or region(s) depicted in the images and, optionally, the views thereof. An individual analyzing an ultrasound image may have difficulty determining whether the particular appearance of a structure in the ultrasound image is due to a characteristic of the subject or some aspect of the procedure by which the ultrasound image was acquired (e.g., difficulty experienced by the ultrasound technician in obtaining a suitable image, the orientation of the ultrasound probe relative to the structure, the configuration of the ultrasound apparatus, etc.). For instance, if an ultrasound probe is oriented such that it images a cylindrical structure at a skew angle, the cross-section of the structure depicted in the image will be oval, rather than circular as would be the case if the probe was oriented to image the structure transversely.

Apparatus for acquiring ultrasound images are becoming both more common and more user-friendly. As a result, ultrasound apparatus may now be found to be used by operators having little or no formal training in the use of ultrasound apparatus and/or anatomy. Where this is the case, it may be desirable to have a record of the use of the ultrasound apparatus as it was used by an operator to acquire an ultrasound image. Since the subject matter of ultrasound examinations may be, without exaggeration, a matter of life and death, it is desirable that such a record can be made without difficulty, and, once made, is readily accessible, comprehensive and reliable.

Often serious consequences turn on diagnoses of medical conditions made using ultrasound images. Where missed or incorrect diagnoses lead to adverse consequences, litigation is often pursued by the adversely affected parties. In such litigation, having a record of the use of the ultrasound apparatus that acquired an ultrasound image used in making a diagnosis may be useful to the parties in discovering the truth of the matter. It may be desirable for doctors, hospitals and liability insurers to have ready access to such records (e.g., from a repository of such records) in the event that such records can demonstrate proper medical conduct. Such records may also be useful in defending or prosecuting lawsuits relating to sexual harassment and termination of employment.

There is a need for practical and cost effective methods and apparatus for providing information useful in the interpretation of ultrasound images.

### BRIEF DESCRIPTION OF DRAWINGS

In drawings that show non-limiting example embodiments:

FIG. 1 is a perspective schematic depiction of an ultrasound examination environment;

FIG. 2 is a schematic illustration of an ultrasound display image according to an example embodiment;

FIG. 3 is a schematic illustration of how an ultrasound image and synthetic display elements may be combined to produce a video signal comprising an ultrasound display image;

FIG. 4 is a flowchart of a method for producing a video record of a use of a medical ultrasound system by storing an ultrasound image video signal and a synthetic display element signal as a single data stream in a multimedia container file according to an example embodiment.

FIG. 5 is a flowchart of a method for producing a video record of a use of a medical ultrasound system by storing an ultrasound image video signal and a synthetic display element signal that contains a set of select synthetic display elements as a single data stream in a multimedia container file according to an example embodiment.

FIG. 6 is a flowchart of a method for producing a video record of a use of a medical ultrasound system by storing an ultrasound image video signal and synthetic display element signal as different video streams in a multimedia container file according to an example embodiment.

FIG. 7 is a flowchart of a method for producing a video record of a use of a medical ultrasound system by storing an ultrasound image video signal and a synthetic display element signal as different data streams in a multimedia container file.

FIG. 8 is a flowchart of a method for producing a video record of a use of a medical ultrasound system by storing an ultrasound image video signal and a subject image video signal as a single data stream in a multimedia container file according to an example embodiment.

FIG. 9 is a flowchart of a method for producing a video record of a use of a medical ultrasound system by storing an ultrasound image video signal, a subject image video signal, and a display element signal as different video data streams in a multimedia container file according to an example embodiment.

FIG. 10 is a flowchart of a method for producing a video record of a use of a medical ultrasound system by storing an ultrasound image video signal and a subject image video signal as a first data stream and storing a display element signal as a second data stream in a multimedia container file according to an example embodiment.

FIG. 11 is a flowchart of a method for producing a video record of a use of a medical ultrasound system by storing an ultrasound image video signal, a subject image video signal, and a display element signal as different data streams in a multimedia container file according to an example embodiment.

FIG. 12 is a flowchart of a method for producing a video record of a use of a medical ultrasound system by storing an ultrasound image video signal as a first data stream and storing a subject image video signal and a display element signal as a second data stream in a multimedia container file according to an example embodiment.

FIG. 13 is a block diagram schematically illustrating a processing apparatus according to an example embodiment.

### DETAILED DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

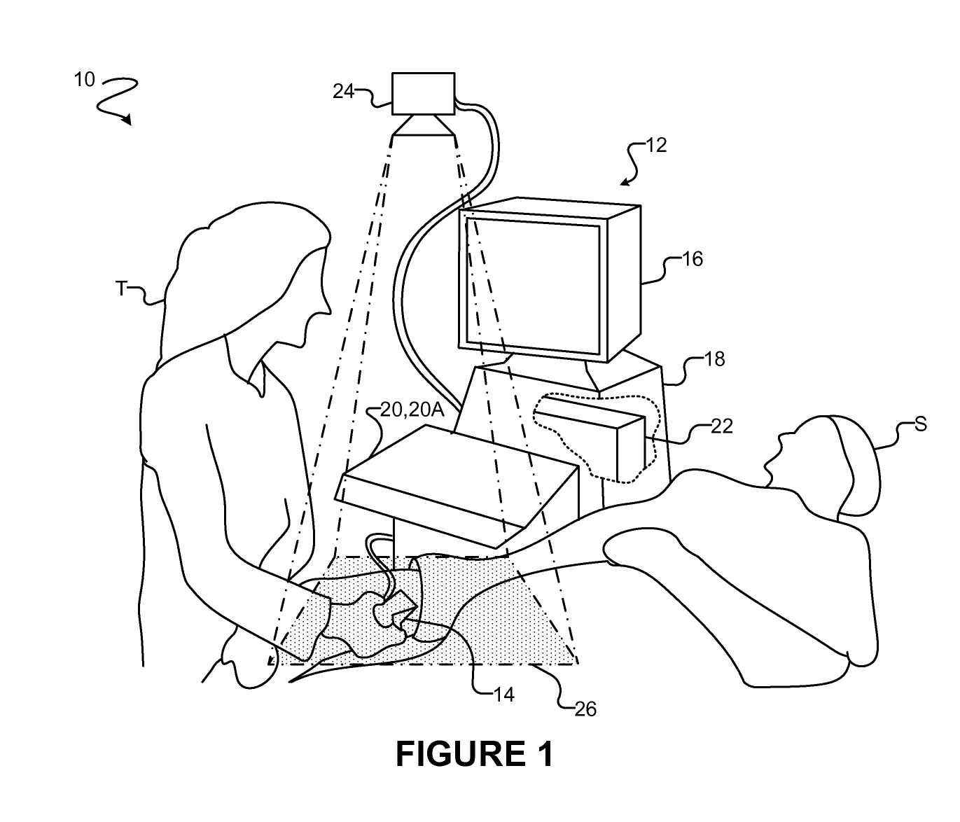

FIG. 1 is a perspective schematic depiction of an ultrasound examination environment 10. An ultrasound technician T uses an ultrasound system 12 to acquire an ultrasound image of a target anatomical structure within a subject S. Ultrasound system 12 comprises a freehand ultrasound probe 14, a display 16, a housing 18, a user interface 20 and a processing apparatus 22. Probe 14 is connected to processing apparatus 22, which is inside housing 18. Processing apparatus 22 may comprise a computer or dedicated ultrasound electronics, and/or the like configured to interface with ultrasound probe 14, for example.

User interface 20 is coupled to processing apparatus 22, and is operable to control the operation of system 12. In the illustrated embodiment, user interface 20 comprises a customized control panel 20A and display 16. User interface 20 may comprise user input interface apparatus such as a keyboard, a computer mouse, a touch screen display, a joystick, a trackball, combinations thereof, or the like, for example. In some embodiments, user interface 20 comprises a graphical user interface displayed on display 16. In some embodiments, user interface 20 comprises user input interface apparatus located on probe 14 (e.g., buttons, switches, a touch screen, and the like). User interface 20 may be operable to configure ultrasound imaging parameters of system 12, such as frequency, depth, gain, focus, compounding, and the like. User interface 20 may provide indications of ultrasound imaging parameters of system 12 (e.g., by displaying visual indications of such parameters on display 16).

In some embodiments, ultrasound system 12 is susceptible to use in one of a plurality of operating modes, each such mode characterized by a plurality of defined ultrasound imaging parameters. In some such embodiments, user interface 20 may be operated to select an operating mode, and may provide an indication of the selected ultrasound mode and/or the ultrasound imaging parameters of system 12 associated therewith.

In use, an ultrasound transducer in probe 14 emits ultrasound pulses which reflect off of structures in the body of subject S (e.g., interfaces where there is a density change in the body of subject S). Reflected ultrasound pulses (echos) are detected at the ultrasound transducer in probe 14. Signals generated by the transducer of probe 14 in response to echos are communicated to processing apparatus 22 inside housing 18. The signals generated in response to echoes comprise timing and strength information, from which processing apparatus 22 constructs images of the structures which caused the echoes. Technician T may maneuver probe 14 over the skin of subject S to acquire different images of structures in the body of subject S.

Ultrasound images generated by processing apparatus 22 may be displayed on display 16. In some embodiments, processing apparatus 22 is configured to provide ultrasound images in an ultrasound image video signal that is provided to display 16. For example, ultrasound processing apparatus may be configured to generate a video signal comprising video frames that include ultrasound images. In some embodiments, system 12 may be operable to acquire and display ultrasound images in real-time or near real-time.

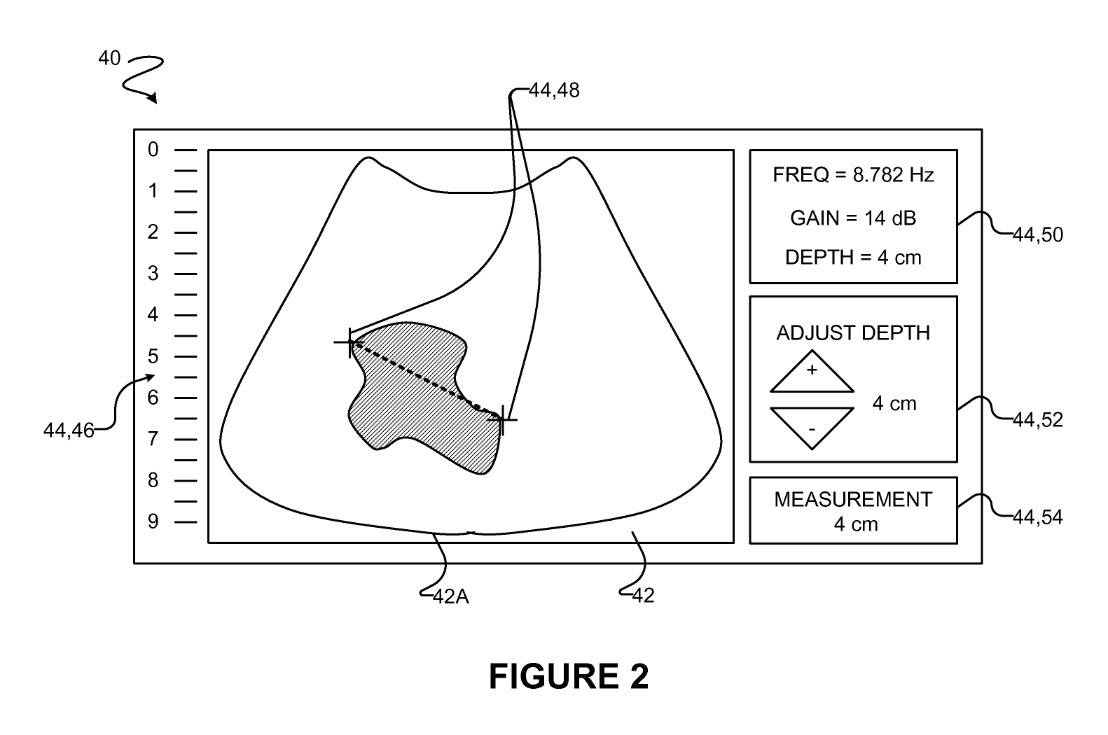

FIG. 2 is a schematic illustration of a display image 40 according to an example embodiment. Display image 40 is representative of display images that may be displayed on display 16. Image 40 comprises an ultrasound image 42 and a plurality of synthetic display elements 44. In the illustrated embodiment, ultrasound image 42 comprises a B-mode image 42A and synthetic display elements 44 comprise a scale 46, a dual-cursor measure 48, a current ultrasound imaging parameter panel 50, a user-interface menu panel 52, and a measurement panel 54. In other embodiments, ultrasound image 42 comprises other types of ultrasound images constructed from ultrasound echo timing and strength information. In other embodiments, synthetic display elements 44 comprise other synthetic display elements 44, such as one or more of subject information, date, time, markers, lines, and the like.

The appearance of ultrasound image 42 may change during operation of system 12. For example, where system 12 acquires and displays ultrasound images in real-time, ultrasound image 42 will change when technician T moves probe 14 to image different parts of the anatomy of subject S. For another example, ultrasound image 42 may be affected by ultrasound imaging parameters controllable via user interface 20.

The appearance of one or more of synthetic display elements 44 may change during operation of ultrasound system 12. For example:
scale 46 may be changed using user interface 20, or may change automatically in response to changes made to ultrasound imaging parameters by use of user interface 20;
one or both cursors of dual-cursor measure 48 may be moved to different locations on ultrasound image element 42 by use of user interface 20;
ultrasound imaging parameter panel 50 displays the current ultrasound imaging parameters, and may change when these parameters are changed by use of user interface 20;
menu panel 52 provides a menu-driven graphical user interface, as part of user interface 20, for interacting with system 12, which interface may change as it is navigated using control panel 20A (e.g., menu panel may change to provide different controls for adjusting ultrasound imaging parameters, viewing subject data, and the like);
measurements panel 54 provides a measurement of a distance spanned by dual-cursor measure 48, which measurement may change as either of cursors of dual-cursor measure 48 is moved by use of user interface 20.

In some embodiments, processing apparatus 22 is configured to produce a video signal comprising display image 40, and the video signal is provided to display 16, which renders the signal as display image 40. Processing apparatus 22 may be configured to produce the video signal by combining an ultrasound image video signal and one or more synthetic display element signals. Combining an ultrasound image video signal and a synthetic display element signal may comprise compositing synthetic display elements and ultrasound images in video frames (e.g., overlaying synthetic display elements on ultrasound images, or vice versa, or a combination of these).

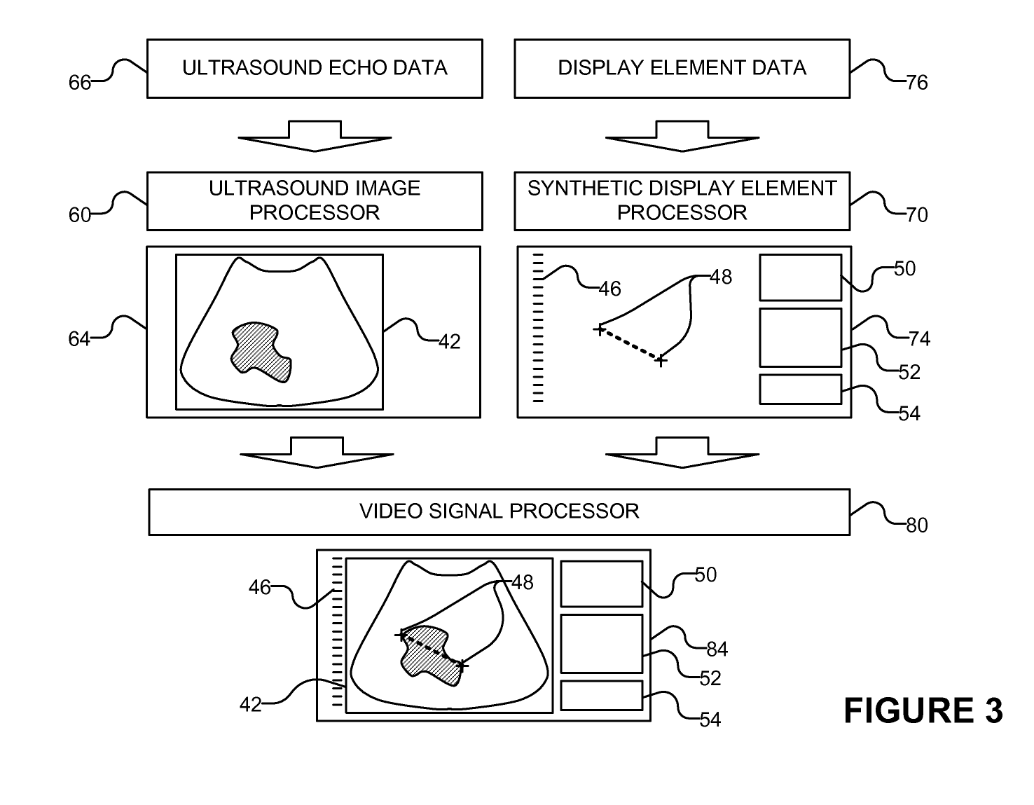

FIG. 3 is a schematic illustration of how an ultrasound image video signal and a synthetic display element signal may be combined to produce a video signal comprising display image 40. An ultrasound image processor 60 comprised in processing apparatus 22 constructs an ultrasound image video signal 64 from ultrasound echo data 66. Ultrasound image video signal 64 comprises at least one ultrasound image (e.g., ultrasound image 42). A synthetic display element processor 70 comprised in processing apparatus 22 generates a synthetic display element signal 74. Display element signal 74 may comprise indications of which display elements are to be displayed, display element source data from which display elements may be generated (e.g., ultrasound imaging parameter values, menu state information, cursor location information, etc.), appearance characteristics of display elements (e.g., size, shape, location, font, color, etc.), and the like. Synthetic display element signal 74 comprises at least one display element (e.g., scale 46, dual-cursor measure 48, ultrasound imaging parameter panel 50, menu panel 52, measurement panel 54, or the like). A video signal processor 80 comprised in processing apparatus 22 combines ultrasound image video signal 64 and synthetic display element signal 74 to produce a video signal 84 that comprises at least one video frame that includes the at least one ultrasound image and the at least one display element.

In some embodiments, display element processor 70 and video signal processor 80 are combined, and display element signal 74 is generated and combined with ultrasound image video signal 64 by modifying ultrasound image video signal 64. In some embodiments, ultrasound image processor 60 and video signal processor 80 are combined, and ultrasound image video signal 64 is generated and combined with display element signal 74 by modifying display element signal 74.

In some embodiments, a plurality of different synthetic display elements are comprised in a corresponding plurality of different synthetic display element signals, and video signal processor 80 combines the plurality of different synthetic display element signals with an ultrasound image video signal to produce a video signal.

In some embodiments, ultrasound system 12 is operable to generate a video record of its use. For example, processing apparatus 22 may be configured to store information pertaining to ultrasound exams conducted using probe 14 in a multimedia container file. In some embodiments, ultrasound system 12 is operable to store an ultrasound image video signal (e.g., ultrasound image video signal 64) and a synthetic display element signal (e.g., synthetic image signal 74) as one or more data streams in a multimedia container file (e.g., a multimedia container file suitable for storing one or more different data streams, such as one or more video streams, audio streams, or the like, as well as synchronization information useful in playing the various streams together) for synchronous playback of the ultrasound image video signal and the synthetic display element signal.

Processing apparatus 22 may comprise a data store that can be used for storing video, such as a non-transitory data recording medium, a memory, a disk, or the like. In some embodiments, ultrasound system 12 comprises a data store external to processing apparatus 22 (e.g., removable media, storage on a server, etc.), and processing apparatus 22 is communicatively coupled to the external memory.

In some embodiments, ultrasound system 12 is operable to store an ultrasound image video signal and a synthetic display element signal as a single data stream in a multimedia container file by combining the ultrasound image video signal and the synthetic display element signal to produce a video signal, encoding the video signal as a video data stream, and storing the video data stream in the multimedia container file. For example, system 12 may be operable to store a video signal provided to display 16 (e.g., a video signal 84 produced by video signal processor 80), which comprises an ultrasound image video signal and a synthetic display element signal. In these embodiments, the stored video signal may comprise the same display images that were viewable by technician T during use of ultrasound system 12. When viewed by medical personnel, the stored video signal may provide information that elucidates the ultrasound images contained in the video signal (e.g., ultrasound imaging parameters indicated by a displayed synthetic display element, ultrasound image video showing a pattern of ultrasound images acquired, etc.).

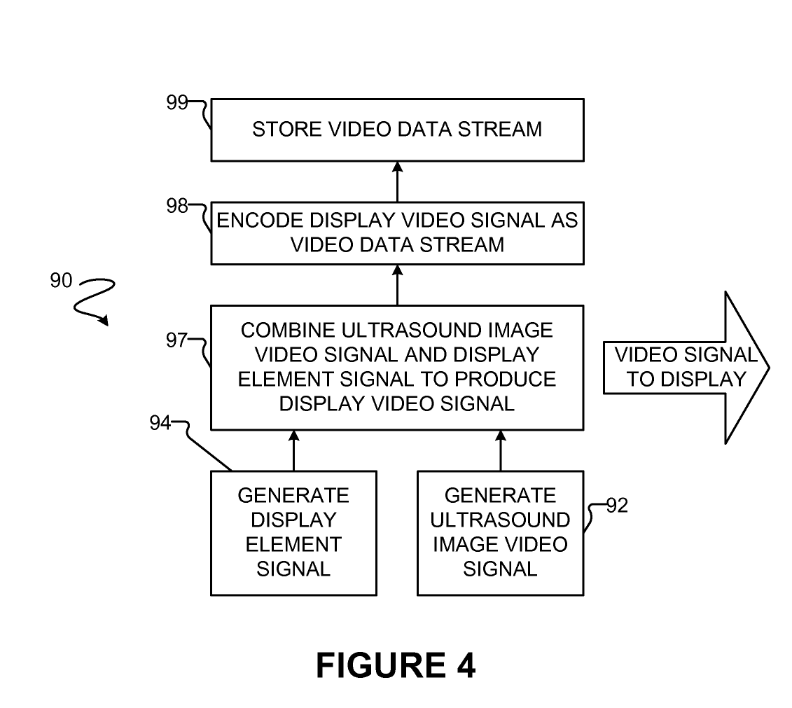

FIG. 4 is a flowchart of a method 90 for producing a video record of a use of a medical ultrasound system by storing an ultrasound image video signal and a synthetic display element signal as a single data stream in a multimedia container file according to an example embodiment. In block 92, an ultrasound image video signal is generated. In block 94, a synthetic display element signal is generated. In block 97, the ultrasound image video signal and synthetic display element signal are combined to produce a display video signal, which may be provided to a display viewable by an ultrasound technician. The display video signal produced in block 97 may be an analog video signal or a digital video signal.

In block 98, the display video signal is encoded as a video data stream. Block 98 may comprise encoding the display video signal with or without data compression. Block 98 may comprise encoding the display video signal using a video codec such as MPEG-4 Part 2 (e.g., DivX™, Xvid™, etc.), H.264/MPEG-4 AVC, MPEG-1 Part 2, H.262/MPEG-2 Part 2, JPEG 2000, Motion JPEG, Windows™ Media Video, Theora™, or the like. In some embodiments, blocks 97 and 98 are combined (e.g., the display video signal provided to the display may be an uncompressed video data stream).

In block 99, the video data stream produced in block 98 is stored. Block 99 may comprise storing the video data stream in its native format. In some embodiments, block 99 comprises storing the video data stream in a multimedia container file, such as an audio-video interleave (AVI) container file, a Matroska (MKV) container file, an MPEG program stream container file, an MPEG transport stream container file, a QuickTime™ multimedia container file, an Adobe™ Flash™ (FLV) container file, an MP4 (or other ISO/IEC 14496-14 based) container file, an Ogg™ container file, a DivX™ media format (DMF) container file, or the like. Block 99 may comprise storing the video data stream in a streaming multimedia container file (i.e., a type of multimedia container file suitable for delivering the video data stream as streaming media). In some embodiments, blocks 98 and 99 are combined. Block 99 may be performed simultaneously with block 98 or after block 98 is complete.

In some circumstances, technician T may wish to record ultrasound images displayed on display 16 without certain synthetic display elements (e.g., display elements that might obscure ultrasound images in the recorded video) or with certain synthetic display element not displayed to technician T (e.g., display elements more useful in interpreting recorded ultrasound images than in assisting technician T in acquiring the images). In some embodiments, user interface 20 may provide a control for selecting a set of computer display elements to be recorded.

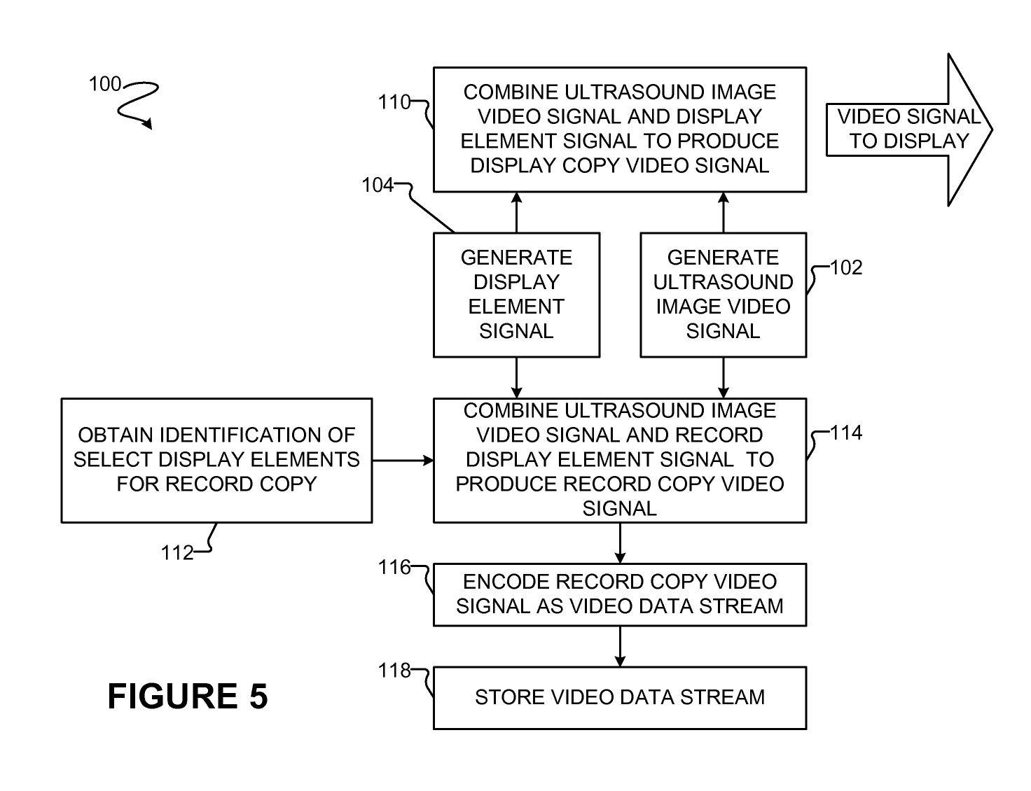

FIG. 5 is a flowchart of a method 100 for producing a video record of a use of a medical ultrasound system by storing an ultrasound image video signal and a synthetic display element signal that contains a set of select synthetic display elements as a single data stream in a multimedia container file according to an example embodiment. In block 102, an ultrasound image video signal is acquired. In block 104, a synthetic display element signal is generated. In block 110, the ultrasound image and synthetic display elements are combined to produce a display copy video signal, which may be provided to a display viewable by an ultrasound technician.

In block 112, an identification of select synthetic display elements to be included in a record copy video signal is obtained. Block 112 may comprise obtaining an identification of select synthetic display elements provided by technician T through user interface 20 (e.g., before the ultrasound image video signal has been acquired, while the ultrasound image video signal is being acquired, or after the ultrasound image video signal has been acquired). The record copy video signal may contain the same display elements displayed during an ultrasound examination or different display elements than were displayed during an ultrasound examination (e.g., less than all display elements displayed during an ultrasound examination, display elements not displayed during an ultrasound examination, etc.).

In block 114, a record display element signal comprising the select synthetic display elements is combined with the ultrasound image video signal to produce a record copy video signal. Block 114 may comprise combining select synthetic display elements with ultrasound images in a manner that replicates or approximates the combining performed in step 110 (e.g., so that display elements common to the display copy elements signal and the record copy video signal are rendered identically or similarly). In some embodiments, block 114 comprises combining the select synthetic display elements with the ultrasound image in a manner that is different from the combining performed in step 110. For example, display elements combined in block 110 to appear adjacent to an ultrasound image in the display copy video signal may be combined in block 114 to appear overlaid on the ultrasound image in the record copy video signal.

In block 116, the record copy video signal is encoded as a video data stream. Block 116 may comprise encoding the record copy video signal using a video codec, including without limitation any of the video codecs mentioned in connection with block 98 of method 90. In some embodiments, blocks 114 and 116 are combined.

In block 118, the video data stream is stored. Block 118 may comprise storing the video data stream in its native format and/or in a multimedia container file, including in any of the multimedia container files mentioned in connection with block 99 of method 90. Block 118 may be performed simultaneously with block 116 or after block 116 is complete.

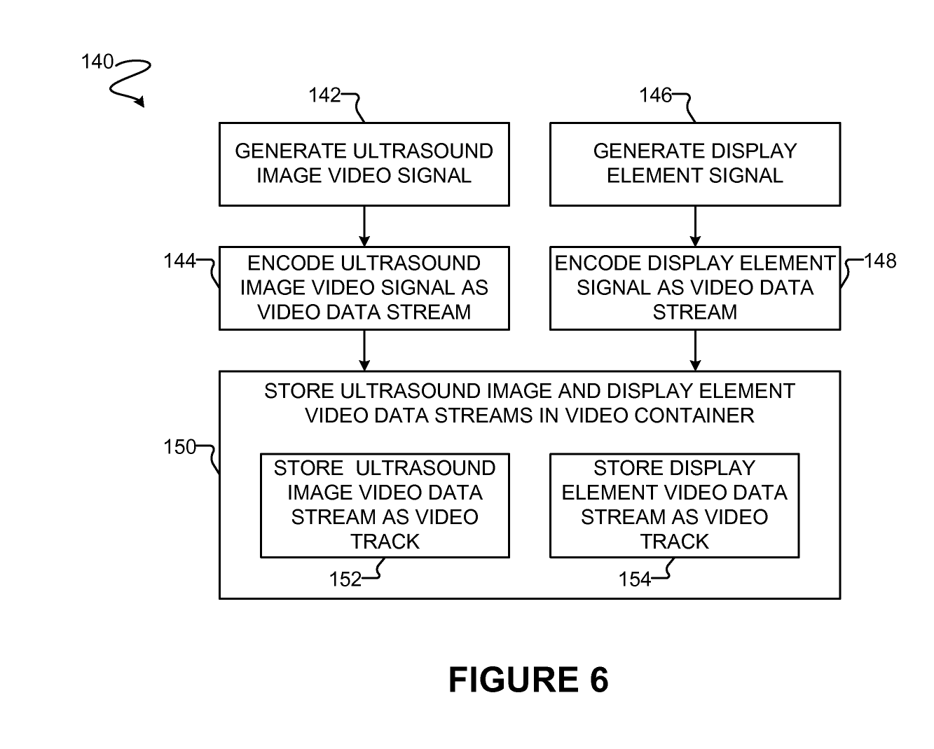

FIG. 6 is a flowchart of a method 140 for producing a video record of a use of a medical ultrasound system by storing an ultrasound image video signal and a synthetic display element signal as different video streams in a multimedia container file according to an example embodiment. In block 142, an ultrasound image video signal is generated. In block 144, the ultrasound image video signal is encoded as a video data stream. Block 144 may comprise encoding the ultrasound image acquired in step 142 using a video codec, including without limitation any of the video codecs mentioned in connection with block 98 of method 90.

In block 146, a synthetic display element signal is generated. In block 148, the display element signal is encoded as a video data stream. Block 148 may comprise rendering the display element signal as raster images and encoding the rendered images as a video data stream. For example, block 148 may comprise rendering display elements contained in the display element signal as pre-rendered subtitles. In some embodiments, block 148 comprises rendering display elements contained in the display element signal as raster images having at least one alpha channel (e.g, a transparent background). For example, block 148 may comprise rendering display elements contained in a synthetic display element signal as non-transparent image features over an alpha channel Block 148 may comprise encoding the display element raster images using a video codec, including any one of the video codecs mentioned in connection with block 98 of method 90.

Blocks 146 and 148 may comprise encoding the ultrasound image video signal and display element signal, respectively, using the same timescale, such that an ultrasound image and associated display elements (e.g., those displayed on display 16 simultaneously with the ultrasound image), will be displayed synchronously when the ultrasound image and display element video data streams are played together.

In block 150, the ultrasound image video data stream and the display element video data stream are stored in a common multimedia container file. Block 150 may be performed simultaneously with block 146 and/or block 148, or may be performed after block 146 and/or block 148 is complete. Block 150 may comprise storing the ultrasound image video data stream and the display element video data stream in any of the multimedia container files mentioned in connection with block 99 of method 90.

Storing separate video streams in a common multimedia container file may comprise assembling the separate video streams according to the format of the multimedia container file. Assembling separate video streams into a multimedia container file may comprise dividing the streams into“chunks” (also know as “atoms”, “packets” and “segments”), and interleaving the chunks. Assembling separate video streams into a multimedia container file may comprise adding metadata, headers and/or synchronization information, for example.

Block 150 comprises sub-blocks 152 and 154. In sub-block 152, the ultrasound image video data stream is stored as a first video track in the multimedia container file. In sub-block 154, the display element video data stream is stored as a second video track in the multimedia container file. In blocks 152 and 154, the second video track may be designated as a higher layer track than the first video track, such that when the tracks are layered for display, the display element video data stream is layered on top of the ultrasound image video data stream.

In some embodiments, block 148 of method 140 comprises encoding the synthetic display element signal as a plurality of different display element video data streams corresponding to the different synthetic display elements contained in the signal. In some such embodiments, block 154 comprises storing the plurality of different display element video data streams in a corresponding plurality of different tracks in the multimedia container file.

Since method 140 provides the ultrasound image video signal and display element signal in different data streams, medical personnel may view the ultrasound image video signal without viewing the display element signal, or may combine (e.g., layer) the ultrasound image video signal and display element signal and view the ultrasound image video signal and display element signal together. For example, a display element video track may be overlaid on an ultrasound image video track to replicate or approximate the appearance of display 16 during an ultrasound examination. It is not necessary that a display element video track be generated such that the display elements thereof are rendered identically or similarly to how they are rendered on the display 16.

Where a multimedia container file comprises a plurality of display element video tracks, medical personnel may combine the ultrasound image video track and select display element video tracks to view the ultrasound image video and select display elements together. In some embodiments, block 150 comprises storing metadata indicative of the display elements displayed on display 16 during an ultrasound examination, and this metadata may be used, for example, to determine a set of select display element video tracks that when overlaid on the ultrasound image video track produces a video that replicates or approximates the appearance of display 16 during the ultrasound examination.

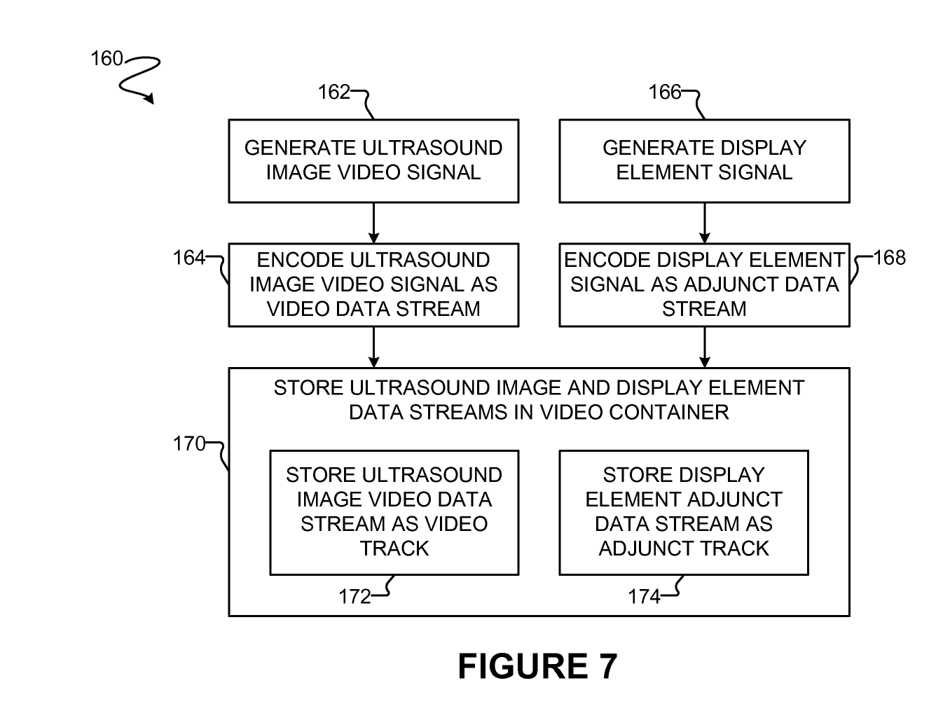

FIG. 7 is a flowchart of a method 160 for producing a video record of a use of a medical ultrasound system by storing an ultrasound image video signal and a synthetic display element signal as different data streams in a multimedia container file. In block 162, an ultrasound image video signal is generated. In block 164, the ultrasound image video signal is encoded as a video data stream. Block 164 may comprise encoding the ultrasound image video signal acquired in step 162 using a video codec, including without limitation any of the codecs mentioned in connection with block 98 of method 90.

In block 166 a synthetic display element signal is generated. In block 168, the display element signal is encoded as an adjunct data stream, such as a raster image stream (e.g., a subpicture stream), a text stream (e.g., a text-based subtitle script), or the like. For example, block 168 may comprise extracting text elements from a display element signal and encoding the text elements as a text-based soft subtitle stream (such as in SubRip Text, Advanced SubStation Alpha, Ogg™ Writ, Structured Subtitle Format, Universal Subtitle Format, XSUB, Binary Information For Scenes (BIFS), 3GPP Timed Text, MPEG-4 Timed Text (TTXT), or like formats). For another example, block 168 may comprise obtaining raster images from a display element signal (e.g., by rendering display elements contained in the display element signal as raster images, by extracting text elements from a display element signal and rendering the text elements as raster images, etc.) and encoding the raster images as a raster image stream (such as subpicture units (e.g., DVD subtitles), as a Blu-Ray™ Presentation Graphic Stream (PGS), in VobSub format, in subpicture file format (SUP), or the like).

Blocks 166 and 168 may comprise encoding the ultrasound image video signal and synthetic display element signal, respectively, using the same timescale, such that an ultrasound image and associated display elements (e.g., the display elements displayed on display 16 simultaneously with the ultrasound image), will be displayed synchronously when the ultrasound image video stream and display element adjunct data streams are played together.

In block 170, the ultrasound image video data stream and the display element adjunct data stream are stored in a single multimedia container file. Block 170 may comprise storing the ultrasound image video data stream and the display element adjunct data stream in any of the multimedia container files mentioned in connection with block 99 of method 90. Block 170 comprises sub-blocks 172 and 174. In sub-block 172, the ultrasound image video data stream is stored as a video track in the multimedia container file. In sub-block 174, the display element adjunct data stream is stored as an adjunct track in the multimedia container file. Sub-block 174 may comprise storing the display element adjunct data stream as a subtitle track, a subpicture track, a presentation graphics track, or the like.

In some embodiments, block 168 comprises encoding information specifying the appearance and/or location of display elements in a frame. Such appearance and location information may be configured to replicate or approximate the appearance and location of display elements on display 16 during an ultrasound examination. For example, block 168 may comprise encoding a raster image of menu panel and information specifying a location of the raster image in a video frame that corresponds to the location of the menu panel on display 16 during an ultrasound examination.

In some embodiments, block 168 comprises encoding the synthetic display element signal as a plurality of different display element adjunct data streams corresponding to the different synthetic display elements contained in the display element signal. In some such embodiments, block 174 comprises storing the plurality of different display element adjunct data streams in a corresponding plurality of different adjunct tracks in the multimedia container file.

Since method 160 provides the ultrasound image video signal and display element signal in different data streams, medical personnel may view the ultrasound image video signal without viewing the display element signal, or may combine (e.g., layer) the ultrasound image video signal and display element signal and view the ultrasound image video signal and display element signal together. For example, a display element adjunct track may be overlaid on an ultrasound image video track to replicate or approximate the appearance of display 16 during an ultrasound examination. It is not necessary that a display element adjunct track be generated such that the display elements thereof are rendered identically or similarly to how they are rendered on the display 16.

Where a multimedia container file comprises a plurality of display element adjunct tracks, medical personnel may combine the ultrasound image video track and select display element adjunct tracks to view the ultrasound image video and select display elements together. In some embodiments, block 170 comprises storing metadata indicative of the display elements displayed on display 16 during an ultrasound examination, and this metadata may be used, for example, to determine a set of select display element adjunct tracks that when overlaid on the ultrasound image video track produces a video that simulates the appearance of display 16 during the ultrasound examination. For example, an association between display elements and display element adjunct tracks may be pre-defined, and a set of select display element adjunct tracks determined from metadata indicative of the display elements displayed on display 16 during an ultrasound examination using a lookup table that embodies the predefined association. For another example, metadata may identify display elements displayed on display 16 during an ultrasound examination by identifiers used to denote corresponding display element adjunct tracks in a multimedia container file.

Returning to FIG. 1, a camera 24 is positioned to capture images of objects in a field of view 26. Camera 24 may be coupled to transmit image data to processing apparatus 22. In some embodiments, camera 24 is operable to provide a subject image video signal to processing apparatus 22, which signal comprises video frames that include images of field of view 26. In some embodiments, the coupling between camera 24 and processing apparatus 22 comprises a wired connection, such as universal serial bus (USB) interface connection, a IEEE 1394 (Firewire™) interface connection, an IEEE 802.3 (Ethernet) connection, or the like, for example. In some embodiments, the coupling between camera 24 and processing apparatus 22 comprises a wireless connection, such as an IEEE 802.11 (Wi-Fi™) connection, a Bluetooth™ connection, a wireless USB connection, or the like, for example.

Non-limiting aspects of the invention include medical ultrasound machines comprising interfaces for receiving video signals from video cameras connected to the interfaces and medical ultrasound machines comprising video cameras and video recording apparatus for recording video images concurrently with performing ultrasound examinations.

In the illustrated ultrasound environment 10, field of view 26 encompasses probe 14, a portion of subject S that includes structures imaged by probe 14, and the hand of technician T used to maneuver probe 14. In some embodiments, field of view 26 is adjustable. For example, camera 24 may be manually repositionable and/or may comprise repositioning actuators (e.g., servo motors, etc.). Camera 24 may comprise zoom functionality (e.g., digital zoom, a zoom lens, etc.).

Camera 24 may be controllable by technician T. In some embodiments, user interface 20 provides a control for the operation of camera 24. For example, user interface 20 may provide a control to change field of view 26 of camera 24, such as by panning and zooming. In some embodiments, probe 14 may comprise a position sensor and system 12 may comprise a position monitoring system (not shown) operable to determine position information indicative of the position of probe 14 in space, and camera 24 may be configured to track the position of probe 14 automatically using the position information determined by the position monitoring system, such that field of view 26 follows probe 14.

In some embodiments, ultrasound system 12 generates a video record of its use that includes subject images acquired by camera 24. For example, processing apparatus may be configured to store an ultrasound image video signal and a subject image video signal as one or more data streams in a multimedia container file for synchronous playback of the ultrasound image video signal and the subject image video signal. When viewed by medical personnel, the one or more data streams may provide information (e.g., position and motion of ultrasound probe 14 relative to subject S in time with ultrasound images, etc.) that elucidates the ultrasound images contained in the data stream(s).

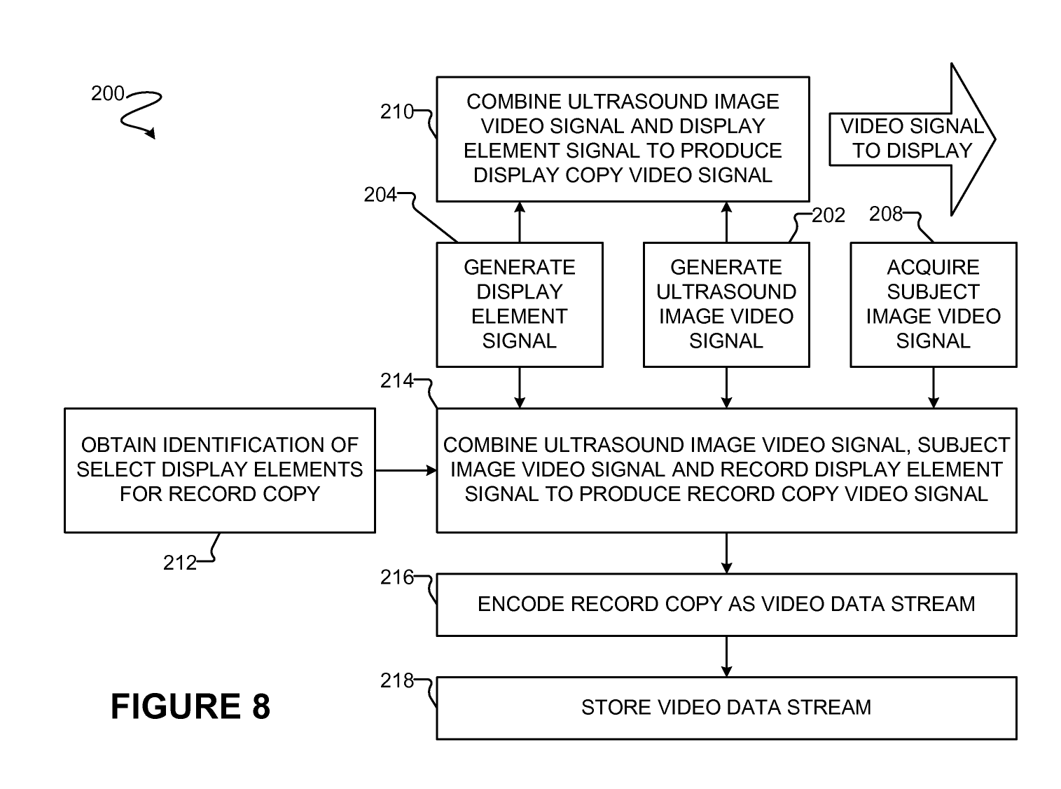

FIG. 8 is a flowchart of a method 200 for producing a video record of a use of a medical ultrasound system by storing an ultrasound image video signal and a subject image video signal as a single data stream in a multimedia container file according to an example embodiment. In block 202, an ultrasound image video signal is generated. In block 204, an output synthetic display element signal is generated. In block 210, the ultrasound image video signal and the output synthetic display element signal are combined to produce a display copy video signal, which may be provided to a display.

In block 208, a subject image video signal is acquired. In optional block 212, an identification of select synthetic display elements to be included in a record copy video signal is obtained. Block 212 may comprise obtaining an identification of select synthetic display elements provided by technician T through user interface 20 (e.g., before recording of video is commenced). In block 214, a record display element signal comprising the select synthetic display elements is combined with the ultrasound image video signal and the subject image video signal to produce a record copy video signal (e.g., by compositing ultrasound images, synthetic display elements and subject images in video frames). Block 214 may comprise overlaying a subject image video signal on an ultrasound image track as a picture-in-picture element, or vice versa, for example.

In embodiments that lack optional step 212, the record display element signal may be the same as the output display element signal, and block 214 comprises combining the output display element signal, the ultrasound image video signal and the subject image video signal. Some such embodiments lack step 210, and the record copy video signal produced in step 14 may be provided to a display.

In block 216, the record copy video signal is encoded as a video data stream. Block 216 may comprise encoding the record copy video signal using a video codec, including without limitation any of the video codecs mentioned in connection with block 98 of method 90. In block 218, the video data stream is stored. Block 218 may comprise storing the video data stream in its native format and/or in a multimedia container file, including in any of the multimedia container files mentioned in connection with block 99 of method 90.

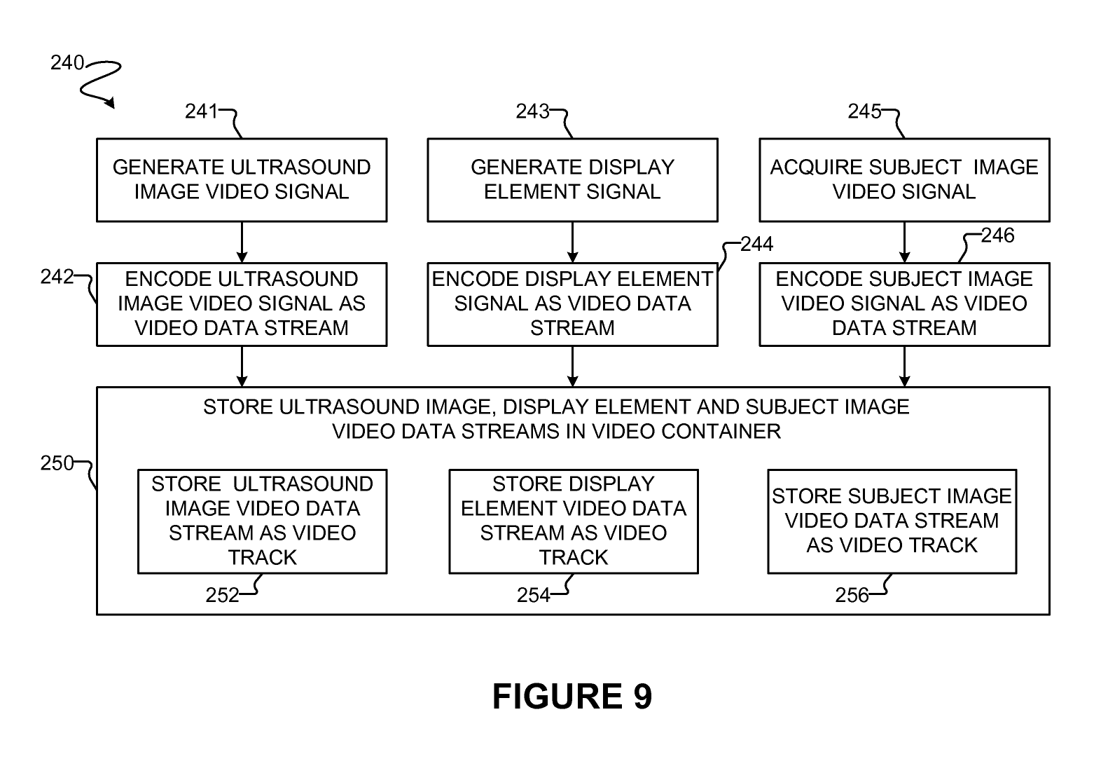

FIG. 9 is a flowchart of a method 240 for producing a video record of a use of a medical ultrasound system by storing an ultrasound image video signal, a subject image video signal, and a display element signal as different video data streams in a multimedia container file according to an example embodiment. In block 241, an ultrasound image video signal is generated. In block 242, the ultrasound image video signal is encoded as a video data stream. Block 242 may comprise encoding the ultrasound image video signal acquired in step 241 using a video codec, including without limitation any of the video codecs mentioned in connection with block 98 of method 90.

In block 243 a synthetic display element signal is generated. In block 244, the display element signal is encoded as a video data stream. Block 244 may comprise rendering display elements contained in the display element signal as one or more raster images and encoding the rendered image as a video stream. For example, block 244 may comprise rendering display elements contained in the display element signal as pre-rendered subtitles. In some embodiments, block 244 comprises rendering display elements contained in the display element signal as one or more raster images over an alpha channel (transparent) background. Block 244 may comprise rendering display elements contained in the display element signal as one or more raster images that replicates or approximates the appearance of the display elements on display 16. Block 244 may comprise encoding the display element signal using a video codec, including any one of the video codecs mentioned in connection with block 98 of method 90.

In block 245, a subject image video signal is acquired. In block 246, the subject image video signal is encoded as a video data stream. Block 246 may comprise encoding the subject image video signal acquired in block 245 using a video codec, including without limitation any of the video codecs mentioned in connection with block 98 of method 90.

Blocks 242, 244 and 246 may comprise encoding the ultrasound image video signal, display element signal and subject image video signal, respectively, using the same timescale, such that an ultrasound image, associated display elements (e.g., those displayed on display 16 simultaneously with the ultrasound image) and an associated subject image (e.g., an image of subject S, technician T and probe 14 acquired at the same time as the ultrasound image was acquired), will be displayed synchronously when the ultrasound image, display element and subject image video data streams are played together.

In block 250, the ultrasound image video data stream, the display element video data stream and the subject image video data stream are stored in a multimedia container file. Block 250 may comprise storing these video streams in any of the multimedia container files mentioned in connection with block 99 of method 90. Block 250 comprises sub-blocks 252, 254 and 256. In sub-block 252, the ultrasound image video data stream is stored as a first video track in the multimedia container file. In sub-block 254, the display element video data stream is stored as a second video track in the multimedia container file. In sub-block 256, the subject image video data stream is stored as a third video track in the multimedia container file. In blocks 252, 254, and 256 the second and third video tracks may be designated as higher layer tracks than the first video track, such that when the video tracks are layered, the display element video data stream and subject image data stream are layered on top of the ultrasound image video data stream.

In some embodiments, block 244 of method 240 comprises encoding a plurality different synthetic display elements as a plurality of corresponding different display element video data streams, and sub-block 254 comprises storing the plurality of different display element video data streams in a corresponding plurality of different tracks in the multimedia container file.

Since method 240 provides the ultrasound image video signal, display element video signal and subject image video signal as different data streams, medical personnel may view the component videos separately (e.g., at different times or simultaneously on different displays), or may combine two or more of the component data streams (e.g., by layering) and view the component videos together on the same display. For example, a display element video track may be overlaid on an ultrasound image video track to simulate the appearance of display 16 during an ultrasound examination. For another example, a subject image video track may be overlaid on an ultrasound image track as a picture-in-picture element.

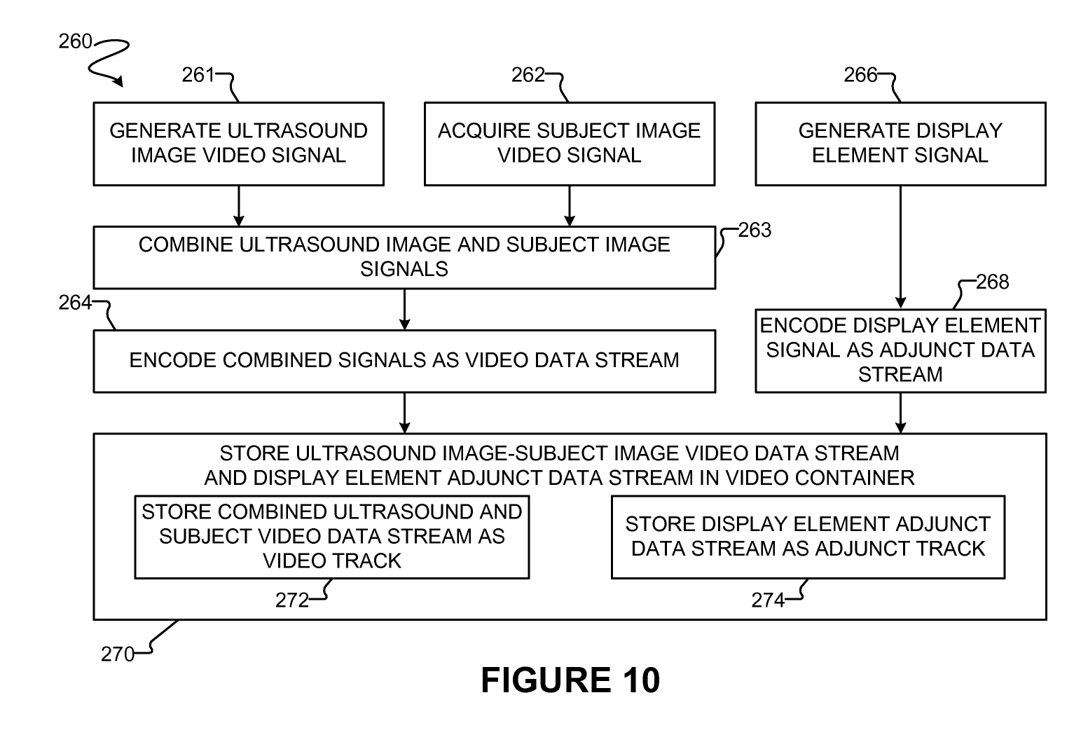

FIG. 10 is a flowchart of a method 260 for producing a video record of a use of a medical ultrasound system by storing an ultrasound image video signal and a subject image video signal as a first data stream and storing a display element signal as a second data stream in a multimedia container file according to an example embodiment. In block 261 an ultrasound image video signal is generated. In block 262, a subject image video signal is acquired. In block 263, the ultrasound image video signal and subject image video signal are combined. Block 263 may comprise overlaying the subject image video signal over the ultrasound image video signal (e.g., as a picture-in-picture element), or may comprise overlaying the ultrasound image video signal over the subject image video signal (e.g., as a picture-in-picture element). In some embodiments, block 263 may comprise non-overlappingly compositing the ultrasound image and subject image video signals (e.g., as a split-screen image signal). In block 264, the combined ultrasound image and subject image video signals are encoded as a video data stream. Block 264 may comprise encoding the combined signal generated in block 263 using a video codec, including without limitation any of the video codecs mentioned in connection with block 98 of method 90.

In block 266, a synthetic display element signal is generated. In block 268, the display element signal is encoded as an adjunct data stream, such as a raster image stream (e.g., a subpicture stream), a text stream (e.g., a text-based subtitle script), or the like. For example, block 268 may comprise extracting and/or generating text from the synthetic display element signal and encoding the text as a text-based soft subtitle stream. For another example, block 268 may comprise rendering the display element signal as raster images and encoding the graphics as a raster image stream.

In some embodiments, block 268 comprises encoding information specifying the appearance and location of graphics elements in a frame. Such appearance and location information may be configured to replicate the appearance and location of display elements on display 16 during an ultrasound examination (e.g., relative to a simultaneously displayed ultrasound image).

Blocks 264 and 268 may comprise encoding the combined image signal and display element signal, respectively, using the same timescale, such that a combined image (e.g., a composite of simultaneous ultrasound and subject images) and associated display elements (e.g., those displayed on display 16 simultaneously with the ultrasound image) will be displayed synchronously when the combined image video data stream and the display element adjunct data stream are played together.

In block 270, the combined image video data stream and the display element adjunct data stream are stored in a multimedia container file. Block 270 comprises sub-blocks 272 and 274. In sub-block 272, the combined image video data stream is stored as a video track in the multimedia container file. In sub-block 274, the display element adjunct data stream is stored as an adjunct track in the multimedia container file. Sub-block 274 may comprise storing the display element data stream as a subtitle track, a subpicture track, a presentation graphics track, or the like.

In some embodiments, block 268 comprises encoding a plurality of different display element signals as a plurality of corresponding different adjunct data streams, and block 274 comprises storing the plurality of different adjunct data streams in a corresponding plurality of different adjunct tracks in the multimedia container file.

As compared with method 240, method 260 may provide output having a smaller memory footprint, since the output of method 260 comprises only one video data stream, rather than two.

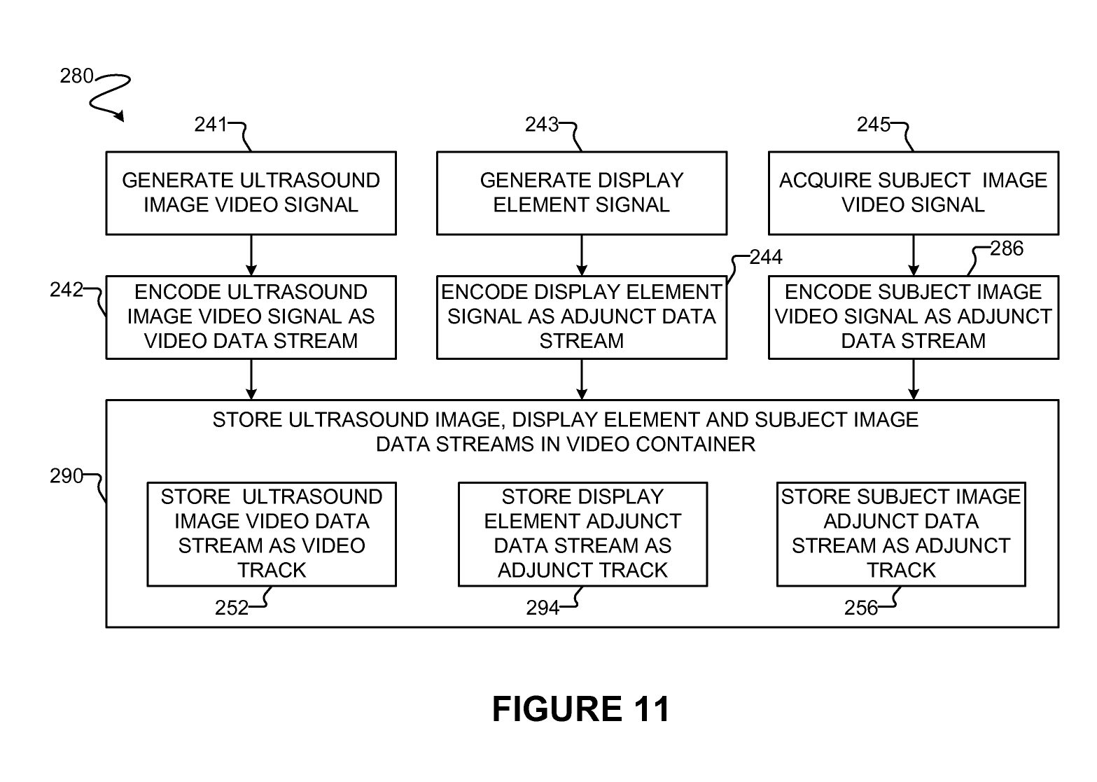

FIG. 11 is a flowchart of a method 280 for producing a video record of a use of a medical ultrasound system by storing an ultrasound image video signal, a subject image video signal, and a display element signal as different data streams in a multimedia container file according to an example embodiment. Method 280 is similar in some respects to method 240 of FIG. 9, and like reference numerals are used to indicate like elements, which are not described again here. Method 280 differs from method 240 in that the subject image video signal acquired in step 245 is encoded as an adjunct data stream (e.g., as a subpicture stream) in step 286, rather than being encoded as a video data stream as in step 246. Step 286 may comprise downsampling, reducing the color depth and/or decreasing frame rate of an acquired subject image video signal (e.g., a subpicture stream may have a lower resolution, lower color depth and lower frame rate than the source subject image video signal).

Method 280 further differs from method 240 in that the step of storing the ultrasound image, display element and subject image streams in the same container file (step 290 in method 280) comprises storing the subject image adjunct data stream as an adjunct track of the multimedia container file (step 294), rather than as a video track (step 254 of method 240).

As compared with method 240, method 280 may provide output having a smaller memory footprint, since the output of method 280 comprises only one video data stream, rather than two.

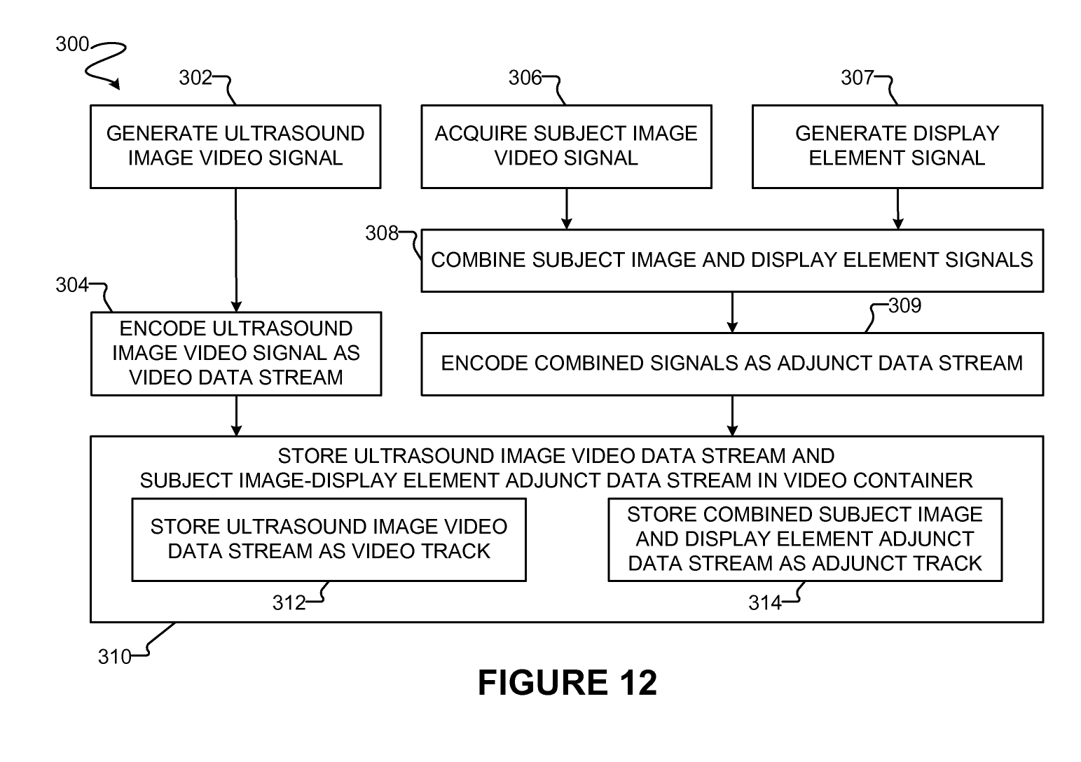

FIG. 12 is a flowchart of a method 300 for producing a video record of a use of a medical ultrasound system by storing an ultrasound image video signal as a first data stream and storing a subject image video signal and a display element signal as a second data stream in a multimedia container file according to an example embodiment. In block 301, an ultrasound image video signal is generated. In block 304, the ultrasound image video signal is encoded as a video data stream. Block 304 may comprise encoding the ultrasound image video signal generated using a video codec, including without limitation any of the video codecs mentioned in connection with block 98 of method 90.

In block 306, a subject image video signal is acquired. In block 307, a synthetic display element signal is generated. In block 308, the subject image video signal and the synthetic display element signal are combined. In some embodiments, block 308 comprises rendering the display elements contained in the display element signal as one or more raster images. In some such embodiments, block 308 may comprise overlaying one or more of the raster images on the subject image video signal and/or non-overlappingly compositing one or more of the raster images with the subject image video signal (e.g., as a split-screen image signal). In block 309, the combined subject image and display element signal is encoded as an adjunct data stream (e.g., a subpicture stream, a presentation graphics stream, etc.).

Blocks 304 and 309 may comprise encoding the ultrasound image video signal and the combined subject image video signal and display element signal, respectively, using the same timescale, such that an ultrasound image and an associated subject image and display elements (e.g., a subject image acquired simultaneously with the ultrasound image and display elements displayed on display 16 simultaneously with the ultrasound image) will be displayed synchronously when the ultrasound image video data stream and the combined subject image and display element adjunct data stream are played together.

In block 310, the ultrasound image video data stream and the combined subject image and display element adjunct data stream are stored in a multimedia container file. Block 310 comprises sub-blocks 312 and 314. In sub-block 312, the ultrasound image video stream is stored as a video track in the multimedia container file. In sub-block 314, the combined subject image and display element adjunct data stream is stored as an adjunct track in the multimedia container file. Sub-block 314 may comprise storing the combined subject image and display element adjunct data stream as a subpicture track, a presentation graphics track, or the like.

As compared with method 280, method 300 may provide output having a smaller memory footprint, since the output of method 300 comprises only one adjunct data stream, rather than two.

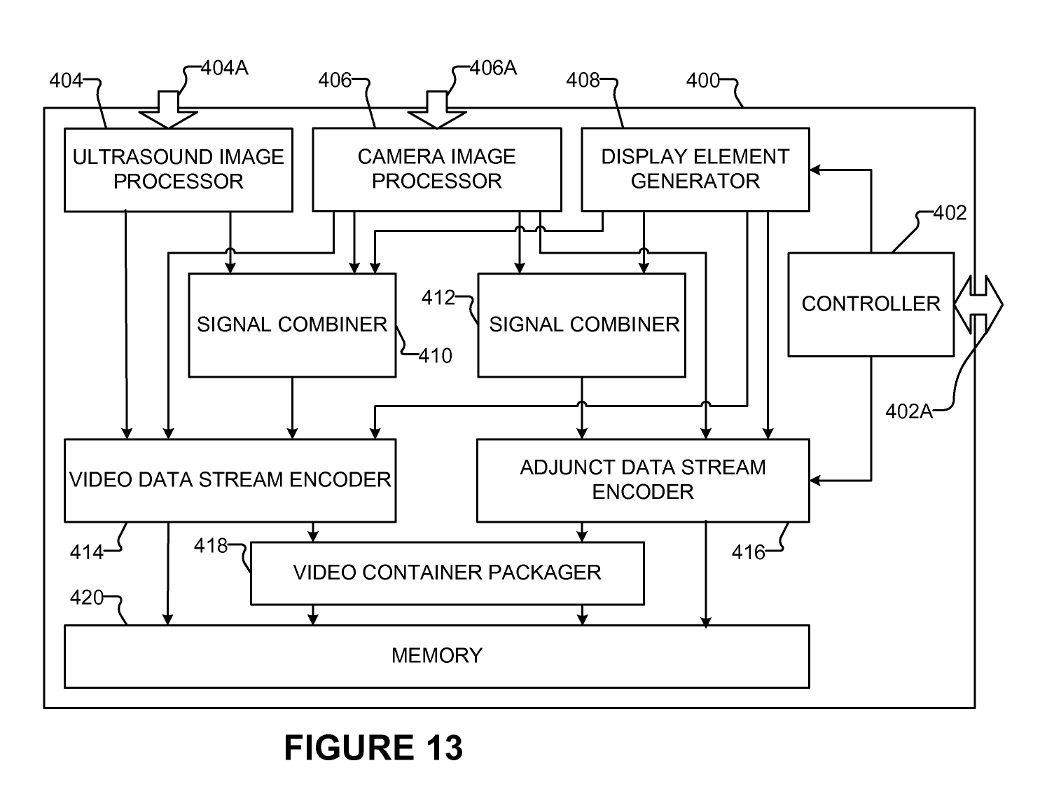

FIG. 13 is a block diagram schematically illustrating a processing apparatus 400 according to an example embodiment. Processing apparatus 400 may be configured to execute one or more of methods 90, 100, 140, 160, 200, 240, 260, 280 and 300. Apparatus 300 comprises a controller 402, an ultrasound image processor 404, a camera image processor 406 and a display element generator 408.

Controller 402 is configured to communicate with user interface 20 via user interface signal 402A. Controller 402 is also configured to control the operation of the components of processing apparatus 400 (e.g., to coordinate performance of steps of methods described herein). Ultrasound image processor 404 is configured to construct ultrasound images from ultrasound echo data 404A, and to generate an ultrasound image video signal containing ultrasound images. Camera image processor 406 is configured to receive image data 406A (e.g., subject image video data) from camera 24, and to produce a subject image video signal. Camera image processor 406 may be configured to process received image data, such as by cropping, zooming, downsampling, modifying color depth, changing frame rate and the like. Display element generator 408 is configured to generate a synthetic display element signal in accordance based on information provided by controller 402.

Apparatus 400 comprises a first signal combiner 410. Ultrasound image processor 404 may be configured to provide an ultrasound image video signal to signal combiner 410. Camera image processor 406 may be configured to provide a subject image video signal to signal combiner 410. Display element generator 408 may be configured to provide a display element signal to signal combiner 410. Signal combiner 410 is configured to produce one or more combined video signals by combining any two or more of a received ultrasound image video signal, subject image video signal and display element signal.

Signal combiner 410 may be configured to combine received signals by layering signals (or portions thereof), by non-overlappingly compositing signals (or portions thereof), by doing combinations of these, and the like. Signal combiner 410 may be configured to convert display elements in a received display element signal into raster images for combining. Signal combiner 410 may be configured to downsample, reduce the color depth and/or decrease the frame rate of received signals and/or images derived from received signals.

Apparatus 400 comprises a second signal combiner 412. Camera image processor 406 may be configured to provide a subject image video signal to signal combiner 412. Display element generator 408 may be configured to provide a display element signal to signal combiner 412. Signal combiner 412 is configured to produce a combined adjunct signal by combining a received subject image video signal and a display element signal. Signal combiner 412 may be configured to combine received signals by layering signals (or portions thereof), by non-overlappingly compositing signals (or portions thereof), by doing combinations of these, and the like. Signal combiner 412 may be configured to convert display elements in a received display element signal into raster images for combining. Signal combiner 412 may be configured to downsample, reduce the color depth and/or decrease the frame rate of received signals and/or images derived from received signals.

In some embodiments, signal combiners 410 and 412 are combined.

Apparatus 400 comprises video data stream encoder 414. Ultrasound image processor 404, camera image processor 406, display element generator 408 and/or signal combiner 410 may be configured to provide video signals to encoder 414. Encoder 414 is configured to encode received video signals as video data streams. Encoder 414 may comprise any of the codecs mentioned in connection with block 98 of method 90, for example. Encoder 414 may be configured to downsample, reduce the color depth and/or decrease the frame rate of received video signals.

Apparatus 400 comprises adjunct data stream encoder 416. Camera image processor 406, display element generator 408 and/or signal combiner 412 may be configured to provide adjunct data signals to encoder 416. Encoder 416 is configured to encode received adjunct data signals as adjunct data streams. Encoder 416 may be configured to encode adjunct data signals containing text or raster images. For example, encoder 416 may be configured to encode a combined subject image and display element adjunct data signal from signal combiner 412 as a raster image stream (e.g., a subpicture stream). For another example, encoder 416 may be configured to encode a display element signal received from display element generator 408 as a text stream (e.g., a text-based subtitle script).

In some embodiments, encoder 416 is configured to extract text elements from a display element signal and encode the text elements as a text-based soft subtitle stream (such as subtitle stream according to any of the formats mentioned in connection with block 168 of method 160). In some embodiments, encoder 416 is configured to obtain raster images from a display element signal (e.g., by rendering display elements contained in the display element signal as raster images, by extracting text elements from a display element signal and rendering the text elements as raster images, etc.) and to encode the raster images as a raster image stream (such as raster stream according to any of the formats mentioned in connection with block 168 of method 160). Encoder 416 may be configured to downsample, reduce the color depth and/or decrease the frame rate of received signals and/or images derived from received signals.

In some embodiments, user interface 20 is operable to acquire narration (e.g., narration provided by an ultrasound technician via a microphone, keyboard, and/or the like) and to provide a narration signal embodying the narration (e.g., in audio and/or text formats). In some such embodiments, such a narration signal may be provided via controller 402 to encoder 416, and encoder 416 is configured to encode the narration stream as an adjunct data stream. For example, encoder 416 may be configured to encode a text-based narration signal as a subtitle data stream. For another example, encoder 416 may be configured to encode an audio narration signal as an audio data stream. Narration provided by an ultrasound technician to explain her actions during an ultrasound examination (e.g., for instructional, diagnostic or other purposes) may thus be encoded for inclusion along with an associated ultrasound image video stream.

Apparatus 400 comprises multimedia container file packager 418. Multimedia container file packager is configured to package encoded data streams received from video data stream encoder 414 or adjunct data stream encoder 416. Multimedia container file packager 418 is configured to package one or more video data streams and/or one or more adjunct data streams in a multimedia container file. Multimedia container file packager 418 may be configured to package video data streams and/or adjunct data streams into any of the container files mentioned in connection with block 99 of method 90, for example.

Apparatus 400 comprises memory 420. Memory 420 may store video data streams, adjunct data streams, and multimedia container files containing one or more video data streams or adjunct data streams.

Variations on the example embodiments disclosed herein are within the scope of the invention, including without limitation:
Methods may comprise obtaining ultrasound examination record data (e.g., patient personal information, case histories, date and time stamps, identifiers of requesting physicians, etc.) and rendering the record data as, or as part of, a video data stream (e.g., as an image overlay on an ultrasound image video signal, display element signal or subject image video signal that is subsequently encoded as a video data stream), and/or as, or as part of, an adjunct data stream and/or storing the record data in a multimedia container file containing an associated ultrasound image signal (e.g., as metadata).
Methods may comprise obtaining high quality still images (e.g., ultrasound images, subject images, etc.) and storing them in memory (e.g., in a container file with video data). For example, the DVD-Video format supports the use of high quality still images that can be displayed for a fixed length of time, or until a defined user input is received. Apparatus may comprise user interface controls for selectively recording high resolution ultrasound images for storage in memory.
Methods may comprise obtaining user-input specifying navigation controls (e.g., menus, chapters, bookmarks etc.) and implementing the user-specified navigation controls in a recorded video (e.g., as DVD-Video menus, DVD-Video data search information, etc.). Apparatus may comprise user interface controls for providing and/or editing navigation controls.
Methods may comprise obtaining user input narration signal (e.g., a text-based narration signal, an audio narration signal, etc.) and adding the user narration to a recorded video (e.g., as an audio track, as a subtitle track, etc.). Apparatus may comprise user interface apparatus for providing and/or controls for editing user narration (e.g., microphones, keyboards, etc.).
Methods may comprise obtaining an ambient audio signal and adding the ambient audio signal to a recorded video (e.g., as an audio track).
User interface 20 may provide controls for recording of information pertaining to ultrasound exams as video by ultrasound system 12. For example, user interface 20 may provide controls for starting recording, ending recording, selecting how ultrasound image video signals and display element signals are stored (e.g., numbers and types of data streams), and the like. Such controls may issue signals that selectively activate apparatus configured to execute blocks of methods disclosed herein. In some embodiments, a control for recording of information pertaining to ultrasound exams as video by ultrasound system 12 comprises a user input apparatus located on probe 14.
User interface 20 may comprise apparatus for acquiring audio signals (e.g., a microphone).

Providing an ultrasound image video signal and a subject image video signal in a single multimedia container file may provide one or more of the following advantages:
Labeling of ultrasound image views is less necessary (if at all), since the ultrasound image view shown in the ultrasound image video signal can be ascertained from the position of probe 14 relative to anatomy of subject S shown in subject image video signal. This may enable personnel untrained in formal descriptions of anatomy to acquire clinically useful ultrasound images.
Errors in labeling ultrasound image views can be detected, since the ultrasound image view shown in the ultrasound image video signal can be ascertained from the position of probe 14 relative to anatomy of subject S shown in subject image video signal.

Providing an ultrasound image video signal and a synthetic display element signal in a single multimedia container file may provide one or more of the following advantages:
Mistakes in measuring anatomical features made by the ultrasound apparatus operator may be detected and possibly compensated for, since display elements used in taking measurements (e.g., cursors) may be viewed, and the instantaneous measurements based on the display elements viewed (e.g., such that the video may be paused when cursors are correctly positioned to take a measurement, and the measurement displayed at that time relied on as a correct measurement).

A single multimedia container file containing an ultrasound image video signal and one or more of a synthetic display element signal and a subject image video signal provides a portable and accessible record of an ultrasound examination, which may be stored using robust and commonly available information management systems and/or streamed over communication networks. Such a record may be useful for:
maintaining medical records (e.g., including measurements obtained during an ultrasound examination which are embedded in video signals contained in the multimedia container file);
supervising ultrasound apparatus operators (e.g., where ultrasound image video signal and a subject image video signal are assembled in a streaming multimedia container file, the signals may be streamed to a physician or other supervisor who are remote from the instructor);
auditing job-performance of ultrasound apparatus operators; and/or
training ultrasound apparatus operators (e.g., where ultrasound image video signal and a subject image video signal are assembled in a streaming multimedia container file, the signals may be streamed to students who are remote from the instructor) and other purposes.

Systems and apparatus described herein may comprise software, firmware, hardware, or any combination(s) of software, firmware, or hardware suitable for the purposes described herein. Software and other modules may reside on servers, workstations, personal computers, computerized tablets, and other devices suitable for the purposes described herein. Those skilled in the relevant art will appreciate that aspects of the system can be practiced with other communications, data processing, or computer system configurations, including: devices (including personal digital assistants (PDAs)), multi-processor systems, microprocessor-based or programmable consumer electronics, mini-computers, mainframe computers, and the like. Furthermore, aspects of the system can be embodied in a special purpose computer or data processor that is specifically programmed, configured, or constructed to implement one or more of the methods, or parts thereof, disclosed herein.

Software and other modules may be accessible via local memory, via a network, via a browser or other application in an ASP context, or via other means suitable for the purposes described herein. Examples of the technology can also be practiced in distributed computing environments where tasks or modules are performed by remote processing devices, which are linked through a communications network, such as a Local Area Network (LAN), Wide Area Network (WAN), or the Internet. In a distributed computing environment, program modules may be located in both local and remote memory storage devices.

Data structures described herein (e.g., data streams, container files, etc.) may comprise computer files, variables, programming arrays, programming structures, or any electronic information storage schemes or methods, or any combinations thereof, suitable for the purposes described herein. User interface elements described herein may comprise elements from graphical user interfaces, command line interfaces, and other interfaces suitable for the purposes described herein.

Image processing and processing steps as described above may be performed in hardware, software or suitable combinations of hardware and software. For example, such image processing may be performed by a data processor (such as one or more microprocessors, graphics processors, digital signal processors or the like) executing software and/or firmware instructions which cause the data processor to implement one or more methods, or parts thereof, disclosed herein. Such methods may also be performed by logic circuits which may be hard configured or configurable (such as, for example logic circuits provided by a field-programmable gate array “FPGA”).

Certain implementations of the invention comprise computer processors which execute software instructions which cause the processors to perform a method of the invention. For example, one or more processors in a processing apparatus or the like may implement methods as described herein by executing software instructions in a program memory accessible to the processors.

The invention may also be provided in the form of a program product. The program product may comprise any non-transitory medium which carries a set of computer-readable signals comprising instructions which, when executed by a data processor, cause the data processor to execute a method of the invention. Program products according to the invention may be in any of a wide variety of forms. The program product may comprise, for example, physical media such as magnetic data storage media including floppy diskettes, hard disk drives, optical data storage media including CD ROMs, DVDs, electronic data storage media including ROMs, flash RAM, hardwired or preprogrammed chips (e.g., EEPROM semiconductor chips), nanotechnology memory, or the like. The computer-readable signals on the program product may optionally be compressed or encrypted. Computer instructions, data structures, and other data used in the practice of the technology may be distributed over the Internet or over other networks (including wireless networks), on a propagated signal on a propagation medium (e.g., an electromagnetic wave(s), a sound wave, etc.) over a period of time, or they may be provided on any analog or digital network (packet switched, circuit switched, or other scheme).

Where a component (e.g. processing apparatus, processor, image processor, signal combiner, data stream encoder, multimedia container file packager, controller, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Where the context permits, words in the above Detailed Description using the singular or plural number may also include the plural or singular number respectively. The word “or,” in reference to a list of two or more items, covers all of the following interpretations of the word: any of the items in the list, all of the items in the list, and any combination of the items in the list.

The above detailed description of examples of the technology is not intended to be exhaustive or to limit the system to the precise form disclosed above. While specific examples of, and examples for, the system are described above for illustrative purposes, various equivalent modifications are possible within the scope of the system, as those skilled in the relevant art will recognize. For example, while processes or blocks are presented in a given order, alternative examples may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, or may be performed at different times.

The teachings of the technology provided herein can be applied to other systems, not necessarily the system described above. The elements and acts of the various examples described above can be combined to provide further examples. Aspects of the system can be modified, if necessary, to employ the systems, functions, and concepts of the various references described above to provide yet further examples of the technology.

These and other changes can be made to the system in light of the above Detailed Description. While the above description describes certain examples of the system, and describes the best mode contemplated, no matter how detailed the above appears in text, the system can be practiced in many ways. Details of the system and method for classifying and transferring information may vary considerably in its implementation details, while still being encompassed by the system disclosed herein. As noted above, particular terminology used when describing certain features or aspects of the system should not be taken to imply that the terminology is being redefined herein to be restricted to any specific characteristics, features, or aspects of the system with which that terminology is associated. In general, the terms used in the following claims should not be construed to limit the system to the specific examples disclosed in the specification, unless the above Detailed Description section explicitly defines such terms. Accordingly, the actual scope of the system encompasses not only the disclosed examples, but also all equivalent ways of practicing or implementing the technology under the claims.

Those skilled in the art will appreciate that certain features of embodiments described herein may be used in combination with features of other embodiments described herein, and that embodiments described herein may be practised or implemented without all of the features ascribed to them herein. Such variations on described embodiments that would be apparent to the skilled addressee, including variations comprising mixing and matching of features from different embodiments, are within the scope of this invention.

As will be apparent to those skilled in the art in the light of the foregoing disclosure, many alterations, modifications, additions and permutations are possible in the practice of this invention without departing from the spirit or scope thereof. The embodiments described herein are only examples. Other example embodiments may be obtained, without limitation, by combining features of the disclosed embodiments. It is therefore intended that the following appended claims and claims hereafter introduced are interpreted to include all such alterations, modifications, permutations, additions, combinations and sub-combinations as are within their true spirit and scope.

What is claimed is:

1. A method for producing a video record of a use of a medical ultrasound system, the medical ultrasound system operable to produce an ultrasound image video signal and a subject image video signal depicting the use of the medical ultrasound system in generating the ultrasound image video signal, the method comprising:
encoding the ultrasound image video signal and the subject image video signal as at least one video data stream; and
storing the at least one video data stream in a multimedia container file for synchronous playback of the ultrasound image video signal and the subject image video signal.

2. The method of claim 1 wherein encoding the ultrasound image video signal and the subject image video signal as at least one video data stream comprises encoding the ultrasound image video signal as a first video data stream and encoding the subject image video signal as a second video data stream, and wherein storing the at least one video data stream in the multimedia container file comprises storing the first and second video data streams in the multimedia container file.

3. The method of claim 1 comprising:
obtaining a text signal from a user interface;
encoding the text signal as a text stream; and
storing the text stream in the multimedia container file for synchronous playback with the ultrasound image video signal.

4. The method of claim 1 comprising:
obtaining an audio signal from a microphone;
encoding the audio signal as an audio data stream; and
storing the audio data stream in the multimedia container file for synchronous playback with the ultrasound image video signal.

5. The method of claim 1 comprising:
obtaining a still ultrasound image acquired by the medical ultrasound system, the still ultrasound image having a resolution higher than a resolution of the ultrasound image video signal;
encoding the still ultrasound image as an adjunct data stream; and
storing the adjunct data stream in the multimedia container.

6. The method of claim 1 wherein the medical ultrasound system is operable to produce a synthetic display element signal, the method comprising encoding the synthetic display element signal as a display element data stream and storing the display element data stream in the multimedia container file for synchronous playback with the ultrasound image video signal.

7. The method of claim 6 wherein the display element data stream comprises an adjunct data stream.

8. The method of claim 1 wherein the medical ultrasound system is operable to produce a synthetic display element signal, the method comprising combining the synthetic display element signal and the ultrasound image video signal, encoding the combined signal as a video data stream, and storing the video stream in the multimedia container file.

9. The method of claim 1 comprising:
obtaining ultrasound examination record data pertaining to an ultrasound examination by which the ultrasound image signal was acquired; and
storing the medical record information as metadata in the multimedia container file.

10. The method of claim 1 comprising:
obtaining ultrasound examination record data pertaining to an ultrasound examination by which the ultrasound image signal was acquired; and
embedding the record data as an image overlay in the ultrasound image video signal.

11. The method of claim 1 comprising:
obtaining ultrasound examination record data pertaining to an ultrasound examination by which the ultrasound image signal was acquired; and
embedding the record data as an image overlay in the subject image video signal.

12. A method for producing a video record of a use of a medical ultrasound system, the medical ultrasound system operable to produce an ultrasound image video signal and a synthetic display element signal, the method comprising:
encoding the ultrasound image video signal and synthetic display element signal as at least one data stream; and
storing the at least one data stream in a multimedia container file for synchronous playback of the ultrasound image video signal and the synthetic display element signal.

13. The method of claim 12 comprising combining the ultrasound image video signal and synthetic display element signal to produce a combined video signal, wherein encoding the ultrasound image video signal and synthetic display element signal as the at least one data stream comprises encoding the combined video signal as a video data stream.

14. The method of claim 12 wherein encoding the ultrasound image video signal and the synthetic display element signal as the at least one data stream comprises encoding the ultrasound image video signal as first data stream and encoding the synthetic display element signal as a second data stream.

15. The method of claim 13 wherein the ultrasound image video signal is encoded as a video data stream.

16. The method of claim 13 wherein the synthetic display element signal is encoded as a video data stream.

17. The method of claim 13 wherein the synthetic display element signal is encoded as an adjunct data stream.

18. The method of claim 13 wherein the synthetic display element signal is encoded as raster image stream.

19. The method of claim 13 wherein the synthetic display element signal is encoded as text stream.

20. The method of claim 12 wherein the medical ultrasound system is operable to produce a subject image video signal depicting the use of the medical ultrasound system in generating the ultrasound image video signal, the method comprising combining the ultrasound image video signal and the subject image video signal to produce a combined video signal, wherein encoding the ultrasound image video signal and the synthetic display element signal as the at least one data stream comprises encoding the combined video signal and the synthetic display element signal as at least one video data stream.

21. The method of claim 14 wherein the medical ultrasound system is operable to produce a subject image video signal depicting the use of the medical ultrasound system in generating the ultrasound image video signal, the method comprising encoding the subject image video signal as a third data stream that comprises a video data stream.

22. The method of claim 12 comprising:
obtaining a text signal from a user interface;
encoding the text signal as a text stream; and
storing the text stream in the multimedia container file for synchronous playback with the ultrasound image video signal.

23. The method of claim 12 comprising:
obtaining an audio signal from a microphone;
encoding the audio signal as an audio data stream; and
storing the audio data stream in the multimedia container file for synchronous playback with the ultrasound image video signal.

24. The method of claim 12 comprising:
obtaining a still ultrasound image acquired by the medical ultrasound system, the still ultrasound image having a resolution higher than a resolution of the ultrasound image video signal;
encoding the still ultrasound image as an adjunct data stream; and
storing the adjunct data stream in the multimedia container.

25. The method of claim 12 wherein the synthetic display element signal comprises at least one display element rendered on a display of the ultrasound system during the use of the ultrasound system.

26. The method of claim 12 comprising:
obtaining ultrasound examination record data pertaining to an ultrasound examination by which the ultrasound image signal was acquired; and
storing the medical record information as metadata in the multimedia container file.

27. The method of claim 12 comprising:
obtaining ultrasound examination record data pertaining to an ultrasound examination by which the ultrasound image signal was acquired; and
embedding the medical record information as an image overlay in the ultrasound image video signal.

28. The method of claim 12 comprising:
obtaining ultrasound examination record data pertaining to an ultrasound examination by which the ultrasound image signal was acquired; and
embedding the medical record information as an image overlay in the subject image video signal.

29. A medical ultrasound system for producing a video record of a use of a medical ultrasound system, the system comprising:
a freehand ultrasound probe;
a camera operable to produce a subject image video signal depicting a use of the probe during the use of the ultrasound medical system;
an ultrasound processing apparatus coupled to receive ultrasound signal information generated by the probe and to receive the subject image video signal from the camera, the ultrasound processing apparatus configured to:
produce an ultrasound image video signal based on the received ultrasound signal information;
encode the ultrasound image video signal and the subject image video signal as at least one video data stream; and
store the at least one video data stream in a multimedia container file for synchronous playback of the ultrasound image video signal and the subject image video signal.

30. The system of claim 29 wherein the ultrasound processing apparatus is configured to:
encode the ultrasound image video signal and the subject image video signal as first and second video data streams, respectively, and store the first and second video data streams in the multimedia container file.
